(19) United States
(12) Patent Application Publication
(10) Pub. No.: US 2026/0191744 A1
(43) Pub. Date: Jul. 9, 2026

(54) SURFACE-MODIFIED DOPED TITANIUM DIOXIDE NANOPARTICLES AND USES

(71) Applicant: THE BOARD OF REGENTS OF THE UNIVERSITY OF OKLAHOMA, Norman, OK (US)

(72) Inventors: Fernando Luis Esteban FLOREZ, Norman, OK (US); Sharukh Soli KHAJOTIA, Norman, OK (US); Adam Justin RONDINONE, Norman, OK (US)

(73) Assignee: THE BOARD OF REGENTS OF THE UNIVERSITY OF OKLAHOMA, Norman, OK (US)

(21) Appl. No.: 19/556,439

(22) Filed: Mar. 4, 2026

Related U.S. Application Data
(62) Division of application No. 17/441,869, filed on Mar. 18, 2020.
(60) Provisional application No. 62/822,161, filed on Mar. 22, 2019.

(51) Int. Cl. A61K 6/84; A61K 6/69; A61K 6/884

(52) U.S. Cl. CPC A61K 6/84; A61K 6/69; A61K 6/884

(57) ABSTRACT

A titanium dioxide (TiO₂) composition and methods of use, the composition containing surface-modified doped TiO₂ nanoparticles (sm-TiO₂ NPs) disposed in a polymer matrix material, wherein each sm-TiO₂ NP has an outer surface having a plurality of bifunctional linker molecules attached thereto and a plurality of protein molecules linked to the sm-TiO₂ NP via the bifunctional linker molecules; and wherein the polymer matrix comprises a polymer precursor component.

### CROSS REFERENCE TO RELATED APPLICATIONS

The present patent application is divisional application of U.S. patent application Ser. No. 17/441,869, entitled “SURFACE-MODIFIED DOPED TITANIUM DIOXIDE NANOPARTICLES AND USES, filed on Sep. 22, 2021, which is U.S. National Phase Application under 35 U.S.C. § 371 of International Patent Application No. PCT/US2020/023377, filed on Mar. 18, 2020, which claims priority under 37 CFR § 119(e) to United States Provisional Patent Application U.S. Ser. No. 62/822,161, filed on Mar. 22, 2019, the entire contents of which are hereby expressly incorporated herein by reference.

### GOVERNMENT SUPPORT

This invention was made with government support under Grant Numbers CNMS18-34 granted by the U. S. Department of Energy, Office of Science, Oak Ridge National Laboratory. The government has certain rights in the invention.

### BACKGROUND

The placement of polymer-based adhesive restorations is one of the most prevalent medical interventions in the human body with more than five hundred million composite restorations placed every year. Resin composite restorations became the first treatment option amongst patients and clinicians around the world due to their outstanding esthetic properties, mercury-free compositions and ultraconservative restorative techniques. Despite their high acceptability and widespread use, these materials have been correlated with significant clinical shortcomings including postoperative sensitivity, shorter service lives, and higher incidences of failure when compared to dental amalgams. The reduced longevity observed has been attributed to a combination of factors including polymerization shrinkage, incomplete enveloping of the dentin matrix and biodegradation. This problem is exacerbated in resin composites and dental adhesive resins, because these materials have been demonstrated to upregulate the aggregation and growth of oral microorganisms, and biofilms accumulated, are typically more cariogenic in nature. Furthermore, it has been suggested that the interface between synthetic and biological materials plays a vital role in shifting the microbial ecology from a state of health into a disease-associated state, which leads to chronic chemical and biological degradation of the tooth-adhesive-resin composite interface and, ultimately, to secondary caries. The occurrence of this biofilm-related disease at the adhesive-tooth interface has consistently been the primary mechanism for failure and replacement of resin composite restorations. It is estimated that a total of $298 billion are spent globally every year for the replacement of failed restorations, which is a heavy economic burden for patients and governments, and represents an average of 4.6% of the total global health-care related expenditures. Several groups have tried to increase the service lives of bonded restorations by adding inhibitors of matrix metalloproteinases (zinc-dependent endopeptidases, MMP), antibacterial agents and monomers to current polymer compositions. Experimental materials containing either chlorhexidine (CHX) or quaternary ammonium compounds (QAC) were previously shown to display promising functionalities (in vitro and in vivo) against a broad variety of oral microorganisms and MMP. However, studies have shown that experimental materials containing these cationic compounds were associated with high levels of water solubility, intense leaching of active compounds and limited long-term antibacterial and anti MMP properties. To overcome this problem, MMP inhibitors conjugated with resin monomers, such as quaternary ammonium dimethacrylates (e.g., 12-methacryloyloxydodecylpyridinium bromide, MDPB) were developed.

Despite substantial efforts from the manufacturing and research communities, none of these materials appear to have vertically advanced the field, because it was found that the incorporation of these hydrophilic monomers into commercially available polymer compositions resulted in experimental materials displaying plasticized polymer matrixes, reduced mechanical properties and increased rates of degradation by hydrolysis. In addition, other studies have demonstrated that saliva adversely impacts the antibacterial activity of QAC-containing experimental materials due to electrostatic interactions between salivary proteins and QAC. Approaches to improve the antibacterial and MMP-inhibiting functionalities of current dental polymer compositions include the utilization of functionalized quaternary ammonium polyethyleneimine nanoparticles (QPEI).

Highly photoactive nitrogen-doped titanium dioxide nanoparticles (N—TiO₂; size distribution 6-15 nm) synthesized by robust solvothermal reactions have been incorporated into dental adhesive resins. The results reported have indicated that experimental dental adhesive resins containing varying concentrations of N—TiO₂ displayed superior antibacterial properties against S. mutans biofilms (3 and 24 hours growth) when compared to the unaltered polymer in both light-irradiated and dark conditions. However, simple incorporation of such nanoparticles into currently-available polymer compositions leads to the attainment of experimental materials with inferior surface, mechanical and biological properties (germicidal, bioactivity and biocompatibility). Consequently, improved compositions of such materials are still needed. It is to addressing this need that the current disclosure is directed.

### BRIEF DESCRIPTION OF THE DRAWINGS

FIG. 1A shows results from Helium-Ion Microscopy analysis (HIM) of unaltered OPTB dental adhesive (field of view=25 μm).

FIG. 1B shows results from HIM of OPTB dental adhesives containing 20% (v/v) of non-doped (n-TiO₂) derivatized with NaOH+APTES+albumin treatment (field of view=25 μm).

FIG. 1C shows results from HIM of OPTB dental adhesives containing 20% (v/v) of nitrogen-doped (N—TiO₂) derivatized with NaOH+APTES+albumin treatment (field of view=25 m).

FIG. 1D shows results from HIM of OPTB dental adhesives containing 20% (v/v) of nitrogen-fluorine-doped (NF-TiO₂) derivatized with NaOH+APTES+albumin treatment (field of view=25 μm).

### DETAILED DESCRIPTION

The simple incorporation of nanoparticles (NPs) into polymers results in materials with inadequate surface properties. Surface-modification and functionalization of the NPs enhances the tooth-adhesive-composite resin interface. In at least one embodiment, the present disclosure includes a TiO₂ composition containing surface-modified doped TiO₂ nanoparticles (sm-TiO₂ NPs) disposed in a curable resin material, wherein each sm-TiO₂ NP has an outer surface, a plurality of bifunctional linker molecules attached to the outer surface, and one or more types of proteins attached to the bifunctional linker molecules. The curable resin material comprises a polymer precursor component. The dopants of the sm-TiO₂ NPs may be, for example, N (nitrogen), Ag (silver), F (fluorine), P (phosphorus), or PO₄ (phosphate), or combinations thereof or other suitable dopants. In certain embodiments, surfaces of doped metaloxide NPs are modified with silanes and proteins to improve the NP-polymer matrix interfaces and to promote self-bonding to organic and inorganic components of the tooth structure.

Experimental results described below demonstrate that the surface-modified NPs were successfully fabricated and covalently functionalized in a commercial adhesive resin, resulting in a material with superior chemical, physical, mechanical and biological properties. This technology can be used in the fields of tissue engineering, dental biomaterials with bioactive properties, bioactive cements for dental and orthopedic fields, 3-D printing of bioactive osteoconductive and osteoinductive materials. The functionalized NPs can be used for the fabrication of antibacterial coatings and paints for use in, for example, hospitals, ambulances, trains, buses, train stations, and airports. These antibacterial coatings and paints may also be used in the naval industries to reduce the formation of marine biofilms, and in and oil and gas industries to reduce corrosive biofilms in oil and gas pipelines, storage tanks, and other containers, machinery, and tools.

Before further describing various embodiments of the compositions and methods of the present disclosure in more detail by way of exemplary description, examples, and results, it is to be understood that the embodiments of the present disclosure are not limited in application to the specific details of methods and compositions as set forth in the following description. The description provided herein is intended for purposes of illustration only and is not intended to be construed in a limiting sense. The inventive concepts of the present disclosure are capable of other embodiments or of being practiced or carried out in various ways. As such, the language used herein is intended to be given the broadest possible scope and meaning; and the embodiments are meant to be exemplary, not exhaustive, and it is not intended that the present disclosure be limited to these particular embodiments. Also, it is to be understood that the phraseology and terminology employed herein is for the purpose of description and should not be regarded as limiting unless otherwise indicated as so. Moreover, in the following detailed description, numerous specific details are set forth in order to provide a more thorough understanding of the disclosure. However, it will be apparent to a person having ordinary skill in the art that the embodiments of the present disclosure may be practiced without these specific details. In other instances, features which are well known to persons of ordinary skill in the art have not been described in detail to avoid unnecessary complication of the description. It is intended that all alternatives, substitutions, modifications and equivalents apparent to those having ordinary skill in the art are included within the scope of the present disclosure. Thus, while the compositions and methods of the present disclosure have been described in terms of particular embodiments, it will be apparent to those of skill in the art that variations may be applied to the formulations, compounds, or compositions and/or methods and in the steps or in the sequence of steps of the method described herein without departing from the spirit and scope of the inventive concepts of the present disclosure.

All patents, published patent applications, and non-patent publications mentioned in the specification are indicative of the level of skill of those skilled in the art to which the present disclosure pertains. Further, all patents, published patent applications, and non-patent publications referenced in any portion of this application are herein expressly incorporated by reference in their entirety to the same extent as if each individual patent or publication was specifically and individually indicated to be incorporated by reference. In particular, International Publication No. WO 2018/106912 A1, which discloses doped and co-doped metal oxide nanoparticles that can be used in the nanoparticle compositions of the present disclosure is hereby expressly incorporated herein by reference in its entirety.

Unless otherwise defined herein, scientific and technical terms used in connection with the present disclosure shall have the meanings that are commonly understood by those having ordinary skill in the art. Further, unless otherwise required by context, singular terms shall include pluralities and plural terms shall include the singular.

As utilized in accordance with the methods and compositions of the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or when the alternatives are mutually exclusive, although the disclosure supports a definition that refers to only alternatives and “and/or.” The use of the term “at least one” will be understood to include one as well as any quantity more than one, including but not limited to, 2, 3, 4, 5, 6, 7, 8, 9, 10, 15, 20, 30, 40, 50, 100, or any integer inclusive therein. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y and Z” will be understood to include X alone, Y alone, and Z alone, as well as any combination of X, Y and Z.

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth. Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-1,000, includes ranges of 1-20, 10-50, 50-100, 100-500, and 500-1,000, for example.

As used in this specification and claims, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

Throughout this application, the term “about” is used to indicate that a value includes the inherent variation of error for the composition, the method used to administer the composition, or the variation that exists among the study subjects. As used herein the qualifiers “about” or “approximately” are intended to include not only the exact value, amount, degree, orientation, or other qualified characteristic or value, but are intended to include some slight variations due to measuring error, manufacturing tolerances, stress exerted on various parts or components, observer error, wear and tear, and combinations thereof, for example. The term “about” or “approximately”, where used herein when referring to a measurable value such as an amount, a temporal duration, and the like, is meant to encompass, for example, variations of ±20% or ±10%, or ±5%, or ±1%, or ±0.1% from the specified value, as such variations are appropriate to perform the disclosed methods and as understood by persons having ordinary skill in the art. As used herein, the term “substantially” means that the subsequently described event or circumstance completely occurs or that the subsequently described event or circumstance occurs to a great extent or degree. For example, the term “substantially” means that the subsequently described event or circumstance (e.g., reaction) occurs at least 90% of the time, or at least 95% of the time, or at least 98% of the time, or to at least 90%, at least 95%, at least 98%, or at least 99% completion.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

The term “pharmaceutically acceptable” refers to compounds and compositions which are suitable for administration to humans and/or animals without undue adverse side effects such as toxicity, irritation and/or allergic response commensurate with a reasonable benefit/risk ratio.

By “biologically active” or “bioactive” is meant the ability to modify or affect the physiological system of an organism without reference to how the active agent has its physiological effects. “Antibacterial” refers to the ability to inhibit the growth of and/or to kill bacteria.

As used herein, “pure,” or “substantially pure” means an object species (e.g., an imaging agent) is the predominant species present (i.e., on a molar basis it is more abundant than any other object species in the composition thereof), and particularly a substantially purified fraction is a composition wherein the object species comprises at least about 50 percent (on a molar basis) of all macromolecular species present. Generally, a substantially pure composition will comprise more than about 80% of all macromolecular species present in the composition, more particularly more than about 85%, more than about 90%, more than about 95%, or more than about 99%. The term “pure” or “substantially pure” also refers to preparations where the object species (e.g., an imaging agent) is at least 60% (w/w) pure, or at least 70% (w/w) pure, or at least 75% (w/w) pure, or at least 80% (w/w) pure, or at least 85% (w/w) pure, or at least 90% (w/w) pure, or at least 92% (w/w) pure, or at least 95% (w/w) pure, or at least 96% (w/w) pure, or at least 97% (w/w) pure, or at least 98% (w/w) pure, or at least 99% (w/w) pure, or 100% (w/w) pure.

The terms “subject” and “patient” are used interchangeably herein and will be understood to refer to a warm-blooded animal, particularly a mammal, and more particularly, humans. Animals which fall within the scope of the term “subject” as used herein include, but are not limited to, dogs, cats, rats, mice, guinea pigs, chinchillas, horses, goats, ruminants such as cattle, sheep, swine, poultry such as chickens, geese, ducks, and turkeys, zoo animals, Old and New World monkeys, and non-human primates.

“Treatment” refers to therapeutic treatments. “Prevention” refers to prophylactic or preventative treatment measures. The term “treating” refers to administering the composition to a patient for therapeutic purposes.

The terms “therapeutic composition,” and “pharmaceutical composition” refer to an active agent-containing composition (e.g., a resin composition comprising doped TiO₂ nanoparticles, such as N—TiO₂ NPs) that may be administered to or used in a subject by any method known in the art or otherwise contemplated herein, wherein administration or use of the composition brings about an effect or result as described elsewhere herein. In addition, the compositions of the present disclosure may be designed to provide delayed, controlled, extended, and/or sustained effects using formulation techniques which are well known in the art.

The term “effective amount” refers to an amount of an active agent (doped TiO₂ nanoparticles) as defined herein (e.g., N—TiO₂ NPs) which is sufficient to exhibit a detectable antibacterial and/or bioactive effect or result without excessive adverse side effects (such as toxicity, irritation and allergic response) commensurate with a reasonable benefit/risk ratio when used in the manner of the inventive concepts. The effective amount for a patient will depend upon the type of patient, the patient's size and health, the nature and severity of the condition to be treated or diagnosed, the method of administration, the duration of treatment, the nature of concurrent therapy (if any), the specific formulations employed, and the like. Thus, it is not possible to specify an exact effective amount in advance. However, the effective amount for a given situation can be determined by one of ordinary skill in the art using routine experimentation based on the information provided herein.

Returning to the particular discussion of the compositions and methods of the present disclosure, as noted above, in certain embodiments, the present disclosure is directed to dental compositions containing doped or co-doped titanium dioxide nanoparticles, such as but not limited to, dental resins, dental bonding agents, dental adhesive resins, dental cements, dental restoratives, dental amalgams, dental bridges, denture bases, dentals coatings, dental sealants, endodontic sealers, guta persha, acrylic resins, denture teeth, dental implants, orthodontic brackets and wires, metallic bands and elastomers and dental bleaching agents for in office and home bleaching techniques. The dental compositions may be used in dentistry, for example, as restorative materials, adhesives, bonding agents, cements, sealants, amalgams, coatings and in the fabrication of partial and complete dentures. These dental compositions have better optical and antibacterial properties when compared to the unaltered commercial dental compositions (e.g., resins) or those comprising undoped TiO₂, and have bioactive properties that can improve the service lives of polymer-based dental biomaterials. These compositions are also anticipated to render better esthetic outcomes (whitening effects) when compared to traditional dental bleaching agents and may allow the utilization of dental bleaching agents displaying ultra-low concentrations of hydrogen peroxide (0.0001% to 5%). The compositions can be used in the fields of tissue engineering, dental biomaterials with bioactive properties, bioactive cements and coatings for implants for dental and orthopedic fields, 3-D printing of bioactive osteoconductive and osteoinductive materials. The functionalized NPs can also be used for the fabrication of self-cleaning and antibacterial coatings and paints for use in public spaces where the control of cross-contamination is important, for example, toilets, hospitals, clinics, private practices, spas and health clubs, ambulances, cars, helicopters, airplanes, passenger trains, buses, cruise-ships, bus stations, train stations, airports, building such as convention centers and gymnasiums or any building in which large numbers of persons meet or congregate, daycare facilities, nursing homes and retirement centers. These antibacterial coatings and paints may also be used in the naval industries to reduce the formation of marine biofilms, and in and oil and gas industries to reduce corrosive biofilms in oil and gas pipelines, storage tanks, and other containers, machinery, and tools. These coatings can also be used for the control of air pollution by being applied to indoor and outdoor surfaces of buildings, sidewalks, and billboards. The presently disclosed antibacterial resins can also be used as antibacterial coatings on furniture, equipment, medical devices and hand-held metallic instruments, catheters, stents, or for imparting antibacterial properties to indoor and outdoor paints.

In a first step after formation of the doped or co-doped NPs, the NPs are treated with a bifunctional linking (linker) molecule such as a silane coupling agent or other linking/coupling compound having at least one functional group (e.g., OH or alkoxy) able to bind to the surface of the TiO₂ particle and at least one functional group (e.g., NH₂) able to bind to a protein. After this treatment, linker molecules decorate the NP outer surface with the protein-binding functional group extending freely from the NP surface. The NPs modified with the bifunctional linking molecule are then treated with a protein which binds to the protein-binding functional group of the linking molecule. The protein is any protein able to bind to the interfaces between the NPs and the polymer matrix in which the surface-modified NPs are combined.

Examples of the bifunctional linking molecules include, but are not limited to, those shown in Table 1. Non-limiting examples of proteins used for binding to the bifunctional linking molecules are shown in Table 2.

TABLE 1
Examples of Bifunctional Linking (Coupling) Molecules

heneicosafluorododecyltrichlorosilane
(3-aminopropyl) triethoxysilane
heptadecafluorodecyltrichlorosilane
poly(tetrafluoroethylene)
octadecyltrichlorosilane
methyltrimethoxysilane
nonafluorohexyltrimethoxysilane
vinyltriethoxysilane
ethyltrimethoxysilane
propyltrimethoxysilane
trifluoropropyltrimethoxysilane
3-(2-aminoethyl)-aminopropyltrimethoxysilane
p-tolyltrimethoxysilane
cyanoethyltrimethoxysilane
aminopropyltrimethoxysilane
acetoxypropyltrimethoxylsilane
phenyltrimethoxysilane
chloropropyltrimethoxysilane
mercaptopropyltrimethoxysilane
glycidoxypropyltrimethoxysilane
γ-methacryloxypropyl trimethoxy silane
vinyl trichlorosilane

TABLE 2
Examples of Linking Proteins

dentin matrix acidic phosphoprotein 1 (DMP1)
integrin-binding site-1
integrin-binding site-2
integrin-binding site-3
osteopontin (OPN)
recombinant human osteopontin (rhOPN)
dentin sialophosphoprotein (DSPP)
matrix extracellular phosphoglycoprotein
(MEPE)

In certain non-limiting embodiments, the compositions of the present disclosure which contain the doped TiO₂ NPs comprise a polymer precursor composition (resin-based matrix), containing least one monomeric component selected from the group: acrylates, methacrylates and dimethacrylates, such as but not limited to ethylenedimethacrylate (EDMA), bisphenol A glycidyl methacrylate (BisGMA), triethyleneglycol dimethacrylate (TEGDMA), 1,6-bis(methacryloxy-2-ethoxycarbonylamino)-2,4,4-trimethylhexane (UDMA), pyromellitic glycerol dimethacrylate (PMGDM), and 2-hydroxyethyl methacrylate (HEMA). The composition may comprise a polymeric material selected from the groups: acrylate resins, methacrylate resins, and dimethacrylate esters resins, epoxy resins, polycarbonate, silicone, polyester, polyether, polyolefin, synthetic rubber, polyurethane, nylon, polystyrene, polyvinylaromatic, polyamide, polyimide, polyvinylhalide, polyphenylene oxide, polyketone, and copolymers and blends thereof. The composition may comprise a solvent selected from the group: water, ethanol, methanol, toluene, ethyl ether, cyclohexane, iso-propanol, chloroform, ethyl acetate hexane, and heptanes. An inorganic filler such as silicon dioxide or glass ceramics. The composition may include a coupling agent such as a silane, a photoinitiator such as camphorquinone (CQ), phenylpropanedione (PPD), or lucirin (TPO) for initiating polymerization, and a catalyst to control the rate of the polymerization reaction.

In non-limiting embodiments, the composition may comprise 0.005 mass % to 10.0 mass % doped TiO₂ NPs (for example, 0.05 mass % to 5.0 mass %, or 0.1 mass % to 2.0 mass %). Other dental compositions which may be modified by the addition of the doped TiO₂ NPs as disclosed herein include, but are not limited to, dental compositions taught in U.S. Pat. Nos. 5,860,806; 7,090,721; and 8,609,741, and US Published Patent Applications, 2009/0023856, 2012/0172485; 2015/0190313; 2016/0058675; and 2016/0228335.

As described in further detail below, nitrogen-doped nanoparticles and nitrogen-fluorine co-doped titanium dioxide nanoparticles (N—TiO₂ NPs, and NF-TiO₂ NPs, respectively) were synthesized using solvothermal reactions. The doped TiO₂ NPs were then subjected to surface modification with either sodium hydroxide (forming NP_{NaOH}), NaOH+(3-Aminopropyl) triethoxysilane (forming NP_{NaOH-APTES}) or NaOH+APTES+albumin (forming NP_{NaOH-APTES}-albumin). The surface-modified NPs were suspended in deuterium oxide (D₂O) containing either NaCl or HCl then were characterized using Small-Angle X-Ray Spectroscopy (SAXS) to determine agglomeration levels of the NPs. Experimental adhesive resins were synthesized by incorporating 20% (v/v) of either n-TiO₂ (undoped TiO₂), N—TiO₂ (nitrogen-doped TiO₂) or NF-TiO₂ (nitrogen and fluorine-doped TiO₂) NPs (as-synthesized or surface-modified) into OptiBond Solo Plus (OPTB, Kerr Corp., USA). Specimens of unaltered (OPTB) and the experimental adhesive resins with the NPs were characterized using Time-of-Flight Secondary Ion Mass Spectroscopy (ToF-SIMS), 2-D ToF-SIMS chemical mapping, Dual Focused Ion-Beam Nano-tomography (Dual-FIBNT) and Small-Angle Neutron Scattering (SANS).

The SAXS results indicated that the surface-modified NPs displayed higher X-Ray scattering intensities in a particle-size dependent manner (NP_{NaOH-APTES}-albumin>NP_{NaOH-APTES}>NP_{NaOH}>NP). ToF-SIMS results demonstrated that NPs incorporation did not adversely impact the polymer structure. 2-D ToF-SIMS chemical mapping determined the Ti⁺ distribution and nitrogen-doping levels. Dual-FIBNT results demonstrated the 3-dimensional distribution of filler particles, NPs and pores within the organic matrix of both unaltered and experimental adhesives. SANS results not only confirmed the NPs' functionalization levels but also determined the types of NP-polymer matrix interfaces.

### EXAMPLES

The present disclosure will now be discussed in terms of several specific, non-limiting, examples and embodiments. The examples described below, which include particular embodiments, will serve to illustrate the practice of the present disclosure, it being understood that the particulars shown are by way of example and for purposes of illustrative discussion of particular embodiments and are presented in the cause of providing what is believed to be a useful and readily understood description of procedures as well as of the principles and conceptual aspects of the present disclosure.

### Materials and Methods

### Synthesis of TiO₂ Nanoparticles

Nanoparticles were synthesized in two steps using very controllable solvothermal reactions. In the first step a solution of 1.7 g of Ti(OBu)₄ (Aldrich, 97%), 4.6 g C₂H₅OH (Decon Labs, 200 proof), 6.8 g C₁₈H₃₅NH₂ (Aldrich, 70%), 7.1 g C₁₈H₃₄O₂(Aldrich, 90%) was prepared and then mixed with an ethanol-water solution (4%, 18-Milli-Q; total volume=20 mL/aliquot). Solutions prepared were transparent before mixing, however, the final solution clouded instantaneously after mixing due to hydrolysis and some micelle formation. Aliquots (20 mL/each) of the final solution were then individually placed into separate high-pressure reaction vessels (Teflon-lined; Paar Series 5000, Multiple Reactor System), reacted (180° C., 24 hours) and stirred via external magnetic field (280 rpm). Room-temperature solutions were then decanted and washed (3×, 200 proof ethanol, Decon Labs) to render pure non-doped TiO₂ nanoparticles (n-TiO₂). A quantity of the n-TiO₂ in ethanol was then reacted (at 140° C., 12 hours) with an equal volume of triethylamine (Sigma-Aldrich, 99.5%) forming nitrogen-doped titanium dioxide nanoparticles (N—TiO₂) were then washed 3 additional times with ethanol, and the concentration of particles was gravimetrically determined to be approximately 40 mg/mL. Nitrogen-fluorine co-doped nanoparticles (NF-TiO₂) were obtained in a single reaction based on step 1 with the inclusion of 5% (wt./wt.; based on Ti content) of fluorine using crystalline Ammonium Fluoride (ACS, 98%, Alfa Aesar) as the dopant source. Aliquots (10 mL/group) of the as-synthesized nanoparticles were re-suspended in deuterium oxide (D₂O, 99.9 atom %, Sigma-Aldrich) in preparation for small-angle X-ray and neutron scattering experiments. Fabrication methods are further disclosed in International patent publication WO 2018/106912.

### Surface Modification of Nanoparticles

As-synthesized nanoparticles (n-TiO₂, N—TiO₂, or NF-TiO₂; ~40 mg/mL) suspended in ethanol (20 mL each) were washed (ultrapure water, 18-Milli-Q, 3 washes, 1 min/wash; 25° C.), centrifuged (8,000 rpm; 3 cycles of 15 min/each) and suspended in a pre-heated sodium hydroxide solution (NaOH, 60° C., 15 M). Ionic solutions containing the nanoparticles were then incubated (30 min.) in an orbital shaker (100 rpm) at room-temperature. Aliquots (10 mL) of NaOH-modified nanoparticles were then centrifuged (8,000 rpm; 3 cycles of 15 min./each) and re-suspended in 20 mL of (3-Aminopropyl) triethoxysilane (APTES; 85.5 mM, Sigma-Aldrich, 99%) at 90° C. for 3 hours (static conditions). Nanoparticles that were surface-modified by NAOH+APTES were then washed and centrifuged as previously described. Silanized nanoparticles were re-suspended in a buffered aqueous solution of human serum albumin (Alb; 10 mg/mL, Sigma-Aldrich, ≥99%, 10% buffer) at room-temperature for 24 hours (100 rpm). Nanoparticles surface-modified (either by NaOH, APTES or Alb; or a combination thereof) were denoted as Dn-TiO₂, DN—TiO₂ or DNF-TiO₂, respectively (where D stands for any type of surface derivatization).

### Small-Angle X-Ray Scattering (SAXS)

Aliquots (10 mL) of the as-synthesized (N—TiO₂,) or surface-modified (DN—TiO₂) nanoparticles were re-suspended in deuterium oxide (D₂O, 99.9 atom %, Sigma-Aldrich) containing either NaCl (0.1 or 1.0 M) or HCl (0.1 M). Aliquots (1.0 mL) of each nanoparticle investigated (either as-synthesized or surface-modified) were then individually placed into separate wells of a disposable plastic sample holder. The SAXS experiment was then performed (8 hours irradiation/sample; 3 samples/group) on a Rigaku BioSAXS-2000 system with a rotating anode, producing CuKα X-ray radiation at 1.54 Å. SAXS data was averaged and reduced using Rigaku SAXSlab data collection and processing software (V4.0.2 Rigaku Americas Corporation).

### Dental Adhesive Resins and Specimen Fabrication

Experimental dental adhesive resins were synthesized by manually dispersing 20% (v/v) of as-synthesized (n-TiO₂, N—TiO₂ or NF-TiO₂) or surface-modified (Dn-TiO₂, DN—TiO₂ or DNF-TiO₂) nanoparticles (in ethanol) into the parental polymer. Disk shaped specimens (n=15/group; diameter=6.0 mm, thickness=0.5 mm) of unaltered (OptiBond Solo Plus (OPTB) Kerr Corp.) or experimental dental adhesive resins (OPTB+20% [v/v] of either n-TiO₂, N—TiO₂, NF-TiO₂ or Dn-TiO₂, DN—TiO₂, DNF-TiO₂) were fabricated by individually pouring uncured materials into the separate wells of a custom-made metallic mold. Specimens were then light-cured with blue light (VALO LED, Ultradent Products, Inc., U.S.A.) from the top (1,000 mW/cm² 60 sec/each).

### Helium Ion Microscopy (HIM)

A helium ion microscope (Zeiss Orion Nanofab) was utilized for the secondary electron imaging of specimens. Helium ion microscopy (HIM), enabled by a gas field ion source (GFIS), is a powerful imaging and nanofabrication technique compatible with many applications in materials science. HIM offers small interaction volume of He and Ne (the two gases offered), small beam spot size, and a moderate sputtering rate. Generally, helium allows higher resolution work, whereas neon offers milling opportunities. Additionally, the HIM can provide sharp, well resolved images from electrically insulating samples (soft, polymeric, and biological substrates) without a conductive coating due to its charge compensation capabilities. In the present work, specimens of each dental adhesive resin investigated were loaded into the vacuum chamber of the HIM at a pressure of ca. 2.5×10⁻⁷ Torr, and GFIS gun pressure was ca. 2×10⁻⁶ Torr. HIM imaging was performed using a focused He⁺ beam with an extraction voltage of 34 kV and acceleration voltage of 25 kV over a range of fields of view (FOV; 2 μm²-100 μm²). The beam current for imaging was measured as ca. 1.65 pA at a beam spot size of 4 μm and a 5 μm gold aperture. Imaging was done for 200 s per pixel dwell time over 1,024×1,024 pixels.

### Time-of-Flight Secondary Ion Mass Spectrometry (ToF-SIMS)

Time-of-flight secondary ion mass spectrometry measurements were carried out using TOF.SIMS.5-NSC instrument (ION-TOF Gmb, Germany) and allowed the surface chemistry characterization of investigated specimens fabricated with unaltered or experimental dental adhesive resins. In ToF-SIMS primary ion beam of Bi₃⁺ clusters with energy of 30 keV, current 30 nA and beam size ~5 μm was used to extract analyte ions from the surface of each specimen. Secondary ions were further accelerated in uniform electric field and moved to the detector. Their time-of-flight was measured and allowed the calculation of mass-to-charge ratios (m/z) and the plotting of full mass spectra. This way ToF-SIMS allowed 2-dimensional chemical imaging of the surface chemistry with mass resolution m/Δm=5,000-10,000 and spatial resolution ~5 μm.

### Small-Angle Neutron Scattering (SANS)

Nanoparticles (either as-synthesized or surface-modified) or specimens fabricated with dental adhesive resins (either unaltered or experimental) were individually placed inside customized titanium cells. Each titanium cell (either containing nanoparticles suspended in D₂O or dry specimens) had two quartz windows to allow the transmission of neutrons through the specimens (nanoparticles in suspension) or samples (unaltered or experimental adhesive resins) investigated. These titanium cells were then individually mounted onto a custom-made and computer-controlled holder (capacity=8 cells/experiment) that allowed the continuous rotation (20 rpm) of individual cells during SANS measurements. The rotation prevented the nanoparticles from settling down in suspension. The SANS experiment was performed (3 hours/sample or specimen) at the Bio-SANS instrument of the High-Flux Isotope Reactor at Oak Ridge National Laboratory. The sample-to-detector distance was set to 15.5 μm (main detector) and 1.13 μm (wing detector) at a wavelength of 6 Å with the wavelength spread Δλ/λ~0.15. The available q range was 0.003<q<0.8 Å⁻¹, where q=((4π sin θ)/λ), and 2θ as the scattering angle. A sample aperture of 12.0 mm diameter was used for providing a sufficient neutron scattering intensity. SANS measurements were taken at room temperature. Raw SANS data were corrected for sample transmission and background radiation by facility supplied reduction software. The data analysis was performed in SASView software (National Science Foundation, DANSE project). A generalized Guinier-Porod function (GPF) was used to fit experimental data of dental adhesive resins (unaltered or experimental) containing 20% (v/v) of nanoparticles (either as-synthesized or surface-modified) according to Equation 1:

$$I ( Q ) = \frac{G}{{Q}^{S}} exp ( \frac{- {Q}^{2} {R}_{g}^{2}}{3 - s} ) \qquad Eq . 1$$

where (G) is a scaling factor, (Rg) is the radius of gyration and (s) is a parameter used to model three-dimensional globular objects (such as spheres or the nanoparticles disclosed herein).

### Results

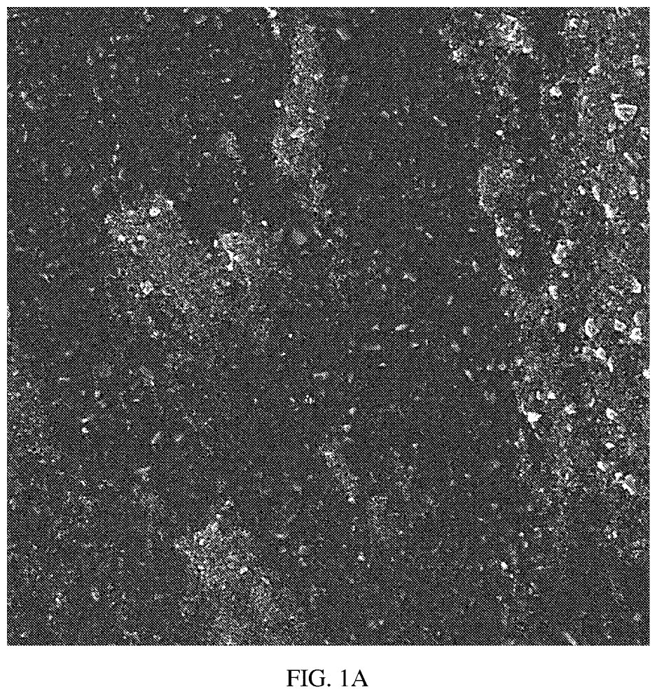

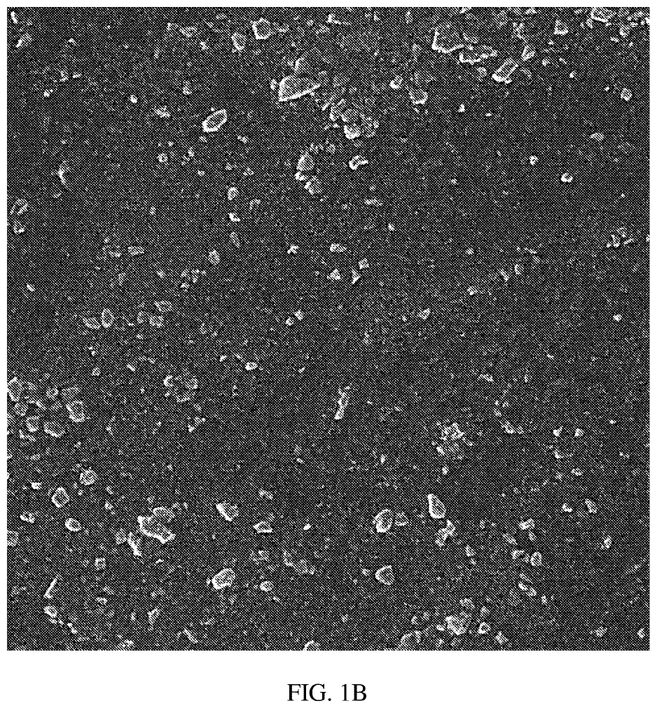

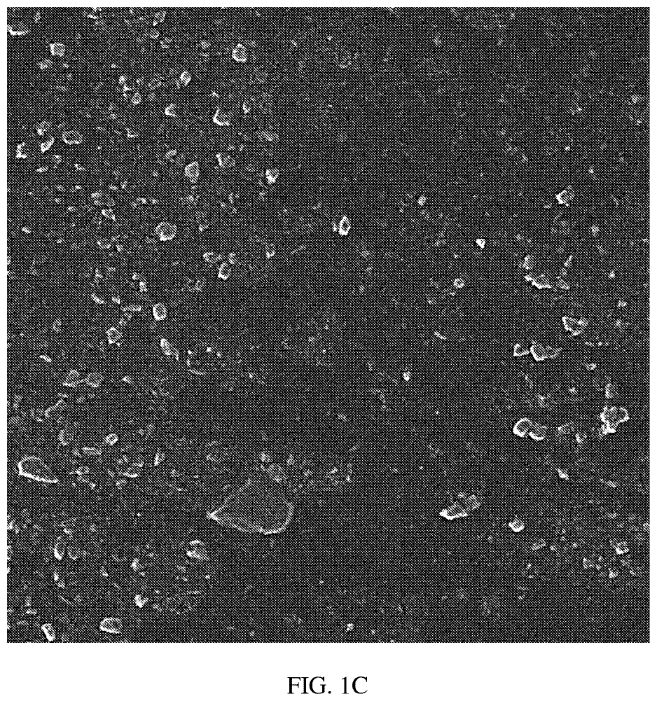

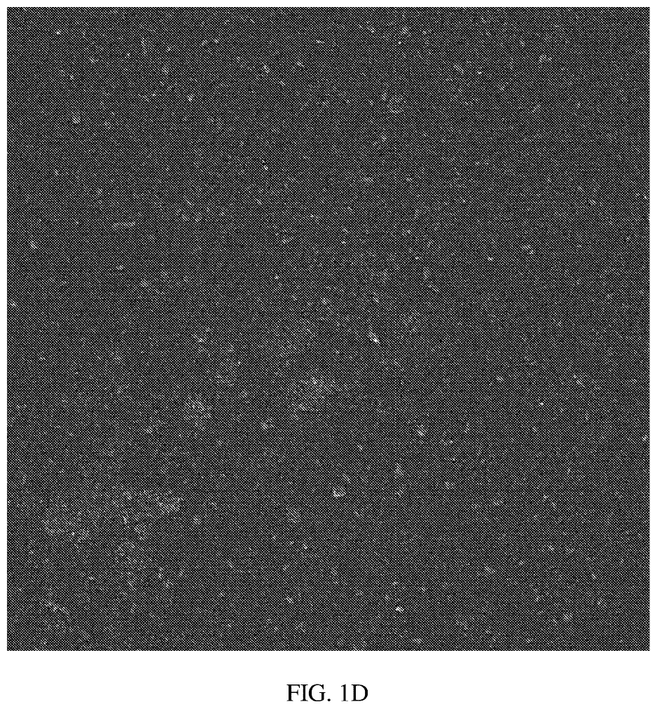

FIGS. 1A-D show results from the surface analysis with HIM (field of view=25 μm²) of unaltered OPTB dental adhesive (FIG. 1A, OPTB) and modified dental adhesive resins containing 20% (v/v) of Dn-TiO₂ (FIG. 1B), DN—TiO₂ (FIG. 1C), and DNF-TiO₂ (FIG. 1D). The modified adhesives had topographical features that were comparable to unaltered OPTB, and phase separation (between the nanoparticles and the organic matrix), at the surface level, was not observed in all groups investigated, thereby suggesting that all nanoparticles investigated (as-synthesized or surface-modified) were successfully incorporated and functionalized within the organic matrix of the presently-modified dental adhesive resins. In addition, it is possible to observe that surface topographies are dominated by the presence of micron-sized filler particles that are typically present in the composition of OptiBond Solo Plus.

SAXS results for N—TiO₂ NPs surface-modified either by sodium hydroxide (NaOH, 15 M), NaOH+(3-Aminopropyl) triethoxysilane (APTES, 85.5 mM), or NaOH+APTES+Albumin (Alb;10 mg/mL) suspended in a solution of deuterium oxide (D₂O) or D₂O containing either NaCl (0.1 or 1.0 M) or HCl (0.1 M) (provided in FIG. 2 of U.S. Provisional Application Ser. No. 822,161) show that for small values of q (between 0.01 and 0.1 Å⁻¹), surface-modified N—TiO₂ were associated with X-ray scattering intensities that were higher in a particle-size dependent manner where NaOH+APTES+Alb>NaOH+APTES>NaOH. These results have not only confirmed the success of the polymer and protein-grafting strategies investigated, but have also indicated for larger values of q (between 0.1 and 1.0 Å⁻¹), that surface modification strategies resulted in nanoparticles displaying higher agglomeration levels. The results also illustrates the impact of the utilization of deuterated ionic solutions (either NaCl or HCl) on the agglomeration behavior of surface-modified nanoparticles, where it was observed that surface-modified nanoparticles suspended in acidic media (HCl 0.1 M) displayed the best isotropic X-ray scattering behavior amongst all experimental groups investigated. These findings indicate that acidic deuterated solutions displayed large quantities of discrete particles (individually distributed) and small-sized agglomerates (15-45 nm in diameter).

ToF-SIMS results of unaltered OPTB and experimental dental adhesive resins containing 20% (v/v) of as-synthesized (n-TiO₂, N—TiO₂ or NF-TiO₂) or surface-modified (Dn-TiO₂, DN—TiO₂ or DNF-TiO₂) nanoparticles (provided in FIG. 3 of U.S. Provisional Application Ser. No. 822,161) show that significant mass spectrum changes were not observed between experimental adhesive resins investigated and OPTB.

Results of 2-D ToF-SIMS chemical imaging (FOV=50 μm²) denoting the distribution of titanium (Ti⁺) within OPTB (4A) and experimental adhesives containing 20% (v/v) of either n-TiO₂ (4B), N_TiO₂ (4C) or NF_TiO₂ (4D) (provided in FIG. 4 of U.S. Provisional Application Ser. No. 822,161) illustrate that the highest amounts of Ti⁺ were observed on specimens containing 20% (v/v) of N_TiO₂, and confirm the findings from the 2-D chemical mapping, and further represent the first instance in dentistry, in which nitrogen-doping is mapped within the crystal lattice of titanium dioxide nanoparticles while immobilized in a commercial adhesive resin.

Small Angle Neutron Scattering (SANS) results for (A)N—TiO₂ (as-synthesized or surface modified) suspended in D₂O (with or without HCl [0.1 M]) or (B) dental adhesive resins (unaltered or experimental) containing 20% of nanoparticles (either n-TiO₂, N—TiO₂, NF-TiO₂, Dn-TiO₂, DN—TiO₂ or DNF-TiO₂) indicated that, for small values of q (Å⁻¹), nanoparticles investigated could be rank ordered in terms of their sizes and agglomeration levels, as follows: N—TiO₂>DN—TiO₂ (NaOH+APTES+Alb)>DN—TiO₂ (NaOH+APTES)>DN—TiO₂ (NaOH+APTES+Alb in HCl [0.1 M]). Table 3 illustrates SANS results for several all dental adhesive resins. The results in Table 3 were used to determine the morphology, size (s), radius of gyration (Rg) of scattering objects and the types of interfaces established between nanoparticles and polymeric chains (Porod exponential). It is possible to observe that radius of gyration (in terms of A) and thickness (in nm) of polymeric chains ranged from 134.99 (N—TiO₂) to 145.16 (Dn-TiO₂), and from 46.7 (N—TiO₂) to 50.2 (Dn-TiO₂), respectively. The results of the s parameter and the Porod exponential (which indicates fractal surface, Table 3) demonstrated the presence of small-sized aggregates (15-50 nm) displaying platelet structures and the establishment of a smooth interface between nanoparticles and the polymeric chains.

TABLE 3
Small Angle Neutron Scattering (SANS) results for OPTB
compositions containing various types of functionalized
and non-functionalized nanoparticles

| | Radius of | | Porod | | Scale |
|---|---|---|---|---|---|
| Groups* | Gyration (Å) | Thickness (nm) | exponential | Background | Factor |
| OPTB only | 142.05 | 49.2 | 3.7 | .28 | .039 |
| n-TiO₂ | 139.37 | 48.3 | 3.7 | .29 | .025 |
| n-TiO₂-APTES-Alb | 145.16 | 50.2 | 3.7 | .24 | .042 |
| N-TiO₂ | 134.99 | 46.7 | 3.7 | .25 | .022 |
| N-TiO₂-APTES-Alb | 140.40 | 48.6 | 3.7 | .28 | .035 |
| NF-TiO₂ | 143.20 | 49.5 | 3.7 | .32 | .039 |
| NF-TiO₂-APTES-Alb | 136.90 | 47.1 | 3.7 | .32 | .025 |

*n-TiO₂-APTES-Alb = TiO₂; N-TiO₂-APTES-Alb = Dn-TiO₂; NF-TiO₂-APTES-Alb = TiO₂.

### Discussion

In the present disclosure undoped (n-TiO₂), doped (N—TiO₂) and co-doped (NF-TiO₂) variations of nanoparticles were synthesized using robust and highly controllable solvothermal reactions. This synthesis route yielded pure and crystalline TiO₂ (anatase phase) displaying high levels of nitrogen-doping in the TiO₂ network when compared to traditional calcination strategies.

We used HIM to characterize the surfaces of non-sputter-coated dental adhesive resins (unaltered or experimental containing 20% [v/v] of either n-TiO₂, N—TiO₂ or NF-TiO₂). The results of the present study indicated that experimental materials investigated had surface characteristics that were comparable to those of the unaltered material (OPTB), and phase separation between nanoparticles and the polymeric matrix could not be observed. The absence of phase separation in nanofilled dental biomaterials is a good indication of successful functionalization of nanoparticles into current polymer compositions, and typically translates into materials with superior physical, mechanical and biological properties. Small-angle X-ray scattering is a non-destructive, powerful and well-established technique in the field of materials science that provides averaged structural data over macroscopic sample volumes. This tool is capable of measuring the structure and size of nanoparticles in situ without the need for removing them from their original environment. SAXS results for surface-modified N—TiO₂ suspended in D₂O or D₂O containing either NaCl (0.1 or 1.0 M) or HCl (0.1 M) indicated that surface-modification strategies investigated were successful in covalently grafting APTES and Alb onto the surfaces of N—TiO₂, as denoted by curves displaying steep slopes and low intensities of X-ray scattering for small values of q (between 0.01 and 0.1 Å⁻¹), thereby suggesting that each surface-modification step implemented, resulted in nanoparticles of slightly larger diameters (NaOH<NaOH+APTES<NaOH+APTES+Alb). In addition to that, the X-ray scattering suggested that surface-modified nanoparticles tend to agglomerate more when compared to non-surfaced derivatized N—TiO₂. Further results indicated that ionic solutions displayed high quantities of discrete nanoparticles and small-sized agglomerates, thereby suggesting that ionic solutions investigated were able to overcome potential negative effects derived from the surface modification strategies implemented.

The control over nanoparticles' agglomeration behavior is anticipated to allow the incorporation of higher fractions of nanoparticles into current dental polymer compositions, and to result in experimental materials displaying superior physical, mechanical and biological properties. ToF-SIMS is a non-destructive and minimally invasive technique that allows for the accurate characterization of complex organic materials with outstanding levels of sensitivity. This remarkable characteristic results from ToF-SIMS' high resolution (spatial and mass) and acquisition speeds (within 0.01 μs), that combined, reduce damage to fragments and recombination of secondary ions. Results from the ToF-SIMS chemical mapping of specimens fabricated with either unaltered or experimental dental adhesive resins containing 20% (v/v) of nanoparticles (as-synthesized or surface-modified) demonstrated that the incorporation of nanoparticles into OPTB did not change the typical ionic fragmentation behavior expected for polymers composed of multifunctional dimethacrylates, which indicates that nanoparticles investigated were compatible with the polymer matrix of OPTB. Results demonstrated the distribution of Ti cations on unaltered or modified adhesive resins containing as-synthesized nanoparticles (n-TiO₂, N—TiO₂ and NF-TiO₂). The highest amounts of Ti⁺ were observed in N—TiO₂.

SANS is an accurate and time-resolved instrument with resolutions at the nanometer and subnanometer levels and, therefore, is considered a powerful tool to investigate the properties of complex materials containing hydrogen. SANS results were obtained for N—TiO₂ (as-synthesized or surface-modified) suspended in D₂O or D₂O containing HCl (0.1 M). The nanoparticles were suspended in D₂O to reduce incoherent background from buffer and to enhance the signal-to-noise ratio. The findings indicated that surface-modification strategies used in the present disclosure were indeed successful in grafting APTES and Alb onto the surfaces of metaloxide nanoparticles. In addition to that, these results corroborate the utilization of low-strength HCl to control nanoparticles' agglomeration prior to their incorporation and functionalization in experimental dental adhesive resins. SANS results demonstrate that all materials investigated displayed neutron scattering behavior that were very similar, which indicates that the incorporation of 20% (v/v) of nanoparticles (either as-synthesized or surface-modified) did not adversely impact the morphology or the structure of polymeric chains in OPTB (in scales from 200-10 nm, correspondent to q ranges between 0.003 and 0.1 Å⁻¹). These findings have further corroborated the results from the HIM and ToF-SIMS analyses regarding the functionalization of nanoparticles in OPTB.

The present disclosure has demonstrated the synthesis (n-TiO₂), doping (e.g., N—TiO₂ or NF-TiO₂) and surface modification (Dn-TiO₂, DN—TiO₂, DNF-TiO₂) of titanium dioxide nanoparticles, as well as, their incorporation into a commercially available dental adhesive resin (OPTB). The present work has shown for the first time in dentistry that surface-modification strategies result in nanoparticles that are larger and tend to display higher agglomeration levels when compared to functionalized N—TiO₂. SAXS and SANS results indicated that low-strength ionic solutions may be used to improve the dispersion of nanoparticles prior to their incorporation into dental adhesive resins. The present work has also demonstrated that the incorporation of nanoparticles (undoped or doped; as-synthesized or surface-modified) did not alter the 3-dimensional lamellar distribution of polymer chains and resulted in experimental materials that did not display any type of phase separation. SANS results also indicated the establishment of smooth interfaces between discrete dispersed nanoparticles and the polymeric matrix, and the covalent functionalization of nanoparticles in OPTB.

While the present disclosure has been described herein in connection with certain embodiments so that aspects thereof may be more fully understood and appreciated, it is not intended that the present disclosure be limited to these particular embodiments. On the contrary, it is intended that all alternatives, modifications and equivalents are included within the scope of the present disclosure as defined herein. Thus the examples described above, which include particular embodiments, will serve to illustrate the practice of the inventive concepts of the present disclosure, it being understood that the particulars shown are by way of example and for purposes of illustrative discussion of particular embodiments only and are presented in the cause of providing what is believed to be the most useful and readily understood description of procedures as well as of the principles and conceptual aspects of the present disclosure. Changes may be made in the formulation of the various compositions described herein, the methods described herein or in the steps or the sequence of steps of the methods described herein without departing from the spirit and scope of the present disclosure. Further, while various embodiments of the present disclosure have been described in claims herein below, it is not intended that the present disclosure be limited to these particular claims.

What is claimed is:

1. A method of treating a dental condition, comprising applying to a subject in need of such dental treatment a titanium dioxide (TiO₂) composition comprising surface-modified doped TiO₂ nanoparticles (sm-TiO₂ NPs) disposed in a polymer matrix material, wherein each sm-TiO₂ NP comprises
an outer surface having a plurality of bifunctional linker molecules attached thereto, and
a plurality of protein molecules linked to the sm-TiO₂ NP via the bifunctional linker molecules, wherein the plurality of protein molecules which are linked to the sm-TiO₂ NP via the bifunctional linker molecules bind to interfaces between the sm-TiO₂ NPs and the polymer matrix material; and
wherein the polymer matrix material comprises a polymer precursor component.

2. The method of claim 1, wherein the sm-TiO₂ NPs comprise one or more dopants selected from the group consisting of N (nitrogen), Ag (silver), F (fluorine), P (phosphorus), and PO₄ (phosphate).

3. The method of claim 1, wherein the bifunctional linker molecule is a silane coupling agent.

4. The method of claim 3, wherein the silane coupling agent is selected from the group consisting of 3-(2-aminoethyl)-aminopropyltrimethoxysilane, heneicosafluorododecyltrichlorosilane, (3-aminopropyl) triethoxysilane, heptadecafluorodecyltrichlorosilane, octadecyltrichlorosilane, methyltrimethoxysilane, nonafluorohexyltrimethoxysilane, vinyltriethoxysilane, ethyltrimethoxysilane, propyltrimethoxysilane, trifluoropropyltrimethoxysilane, p-tolyltrimethoxysilane, cyanoethyltrimethoxysilane, aminopropyltrimethoxysilane, acetoxypropyltrimethoxylsilane, phenyltrimethoxysilane, chloropropyltrimethoxysilane, mercaptopropyltrimethoxysilane, glycidoxypropyltrimethoxysilane, γ-methacryloxypropyl trimethoxysilane, and vinyl trichlorosilane.

5. The method of claim 1, wherein the plurality of protein molecules is selected from the group consisting of dentin matrix acidic phosphoprotein 1 (DMP1), integrin-binding site-1, integrin-binding site-2, integrin-binding site-3, osteopontin (OPN), recombinant human osteopontin (rhOPN), dentin sialophosphoprotein (DSPP), and matrix extracellular phosphoglycoprotein (MEPE).

6. The method of claim 1, wherein the polymeric matrix material is selected from the group consisting of acrylate resins, methacrylate resins, dimethacrylate ester resins, epoxy resins, polycarbonate, silicone, polyester, polyether, polyolefin, synthetic rubber, polyurethane, nylon, polystyrene, polyvinylaromatic, polyamide, polyimide, polyvinylhalide, polyphenylene oxide, polyketone, and copolymers and blends thereof.

7. The method of claim 1, wherein the polymer precursor component comprises at least one monomeric component selected from the group consisting of acrylates, methacrylates and dimethacrylates.

8. The method of claim 7, wherein the polymer precursor component comprises at least one monomeric component selected from the group consisting of ethylenedimethacrylate (EDMA), bisphenol A glycidyl methacrylate (BisGMA), triethyleneglycol dimethacrylate (TEGDMA), 1,6-bis(methacryloxy-2-ethoxycarbonylamino)-2,4,4-trimethylhexane (UDMA), pyromellitic glycerol dimethacrylate (PMGDM), and 2-hydroxyethyl methacrylate (HEMA).

9. The method of claim 1, wherein the polymer matrix material is a curable resin material.

10. The method of claim 9, wherein the curable resin material is a dental material selected from the group consisting of dental resins, dental bonding agents, dental adhesive resins, dental cements, dental restoratives, dental amalgams, dental bridges, denture bases, dental coatings, dental sealants, endodontic sealers, guta persha, acrylic resins, denture teeth, and dental implants.

11. A method of treating a dental condition, comprising applying to a subject in need of such dental treatment a titanium dioxide (TiO₂) composition, comprising surface-modified doped TiO₂ nanoparticles (sm-TiO₂ NPs) disposed in a polymer matrix material, wherein each sm-TiO₂ NP comprises
an outer surface having a plurality of bifunctional linker molecules attached thereto, and
a plurality of protein molecules linked to the sm-TiO₂ NP via the bifunctional linker molecules, wherein the plurality of protein molecules which are linked to the sm-TiO₂ NP via the bifunctional linker molecules bind to interfaces between the sm-TiO₂ NPs and the polymer matrix material; and
wherein the polymer matrix material comprises a polymer precursor component; and wherein
(a) the sm-TiO₂ NPs comprise one or more dopants selected from the group consisting of N (nitrogen), Ag (silver), F (fluorine), P (phosphorus), and PO₄ (phosphate),
(b) the bifunctional linker molecule is a silane coupling agent selected from the group consisting of 3-(2-aminoethyl)-aminopropyltrimethoxysilane, heneicosafluorododecyltrichlorosilane, (3-aminopropyl) triethoxysilane, heptadecafluorodecyltrichlorosilane, octadecyltrichlorosilane, methyltrimethoxysilane, nonafluorohexyltrimethoxysilane, vinyltriethoxysilane, ethyltrimethoxysilane, propyltrimethoxysilane, trifluoropropyltrimethoxysilane, p-tolyltrimethoxysilane, cyanoethyltrimethoxysilane, aminopropyltrimethoxysilane, acetoxypropyltrimethoxylsilane, phenyltrimethoxysilane, chloropropyltrimethoxysilane, mercaptopropyltrimethoxysilane, glycidoxypropyltrimethoxysilane, γ-methacryloxypropyl trimethoxysilane, and vinyl trichlorosilane,
(c) the polymeric matrix material is selected from the group consisting of acrylate resins, methacrylate resins, dimethacrylate ester resins, epoxy resins, polycarbonate, silicone, polyester, polyether, polyolefin, synthetic rubber, polyurethane, nylon, polystyrene, polyvinylaromatic, polyamide, polyimide, polyvinylhalide, polyphenylene oxide, polyketone, and copolymers and blends thereof, and
(d) the polymer precursor component comprises at least one monomeric component selected from the group consisting of acrylates, methacrylates and dimethacrylates.
